(19) United States
(12) Patent Application Publication
(10) Pub. No.: US 2008/0187894 A1
(43) Pub. Date: Aug. 7, 2008

(54) Method and System for Interactive Cognitive Testing

(76) Inventor: Roger K. Cady, Ozark, MO (US)

Correspondence Address: HUSCH BLACKWELL SANDERS LLP, 190 CARONDELET PLAZA, SUITE 600, ST. LOUIS, MO, 63105-3441, US

(21) Appl. No.: 11/956,580

(22) Filed: Dec. 14, 2007

Related U.S. Application Data
(60) Provisional application No. 60/869,985, filed on Dec. 14, 2006.

(51) Int. Cl. G09B 19/00; C12M 1/34

(52) U.S. Cl. 434/236; 435/287.2

(57) ABSTRACT

A cognitive testing and assessment system includes a testing module, said testing module having an input device, a memory, and at least one cognitive test battery stored in said memory, a wireless communication module, and an assessment and response module. The wireless communication module allows the system to communicate with external devices or networks, such as the Internet, local area networks, wide area networks, satellites, or cellular networks, without a wired connection. The assessment and response center may be either integrated into the system or located in a remote centralized or regional facility that the system accesses via the wireless communication module.

### CROSS REFERENCE

This application claims the priority of provisional application Ser. No. 60/869,985, filed Dec. 14, 2006.

### BACKGROUND OF THE INVENTION

The present invention relates generally to the health field and more particularly to a method and system for wireless, interactive cognitive testing, assessment, and response.

### BACKGROUND OF THE INVENTION

Cognitive testing methodologies have been developed to determine changes in cognitive efficiency of individuals. In particular, objective cognitive tests have shown significant promise of providing a standard and repeatable testing methodology. These objective cognitive testing methodologies have been incorporated into computerized testing systems, and in some cases, even portable, handheld systems.

However, the prior art systems to date have been stand-alone units that lack interactive capability and are unable to independently take appropriate action in the event that a user suffers a significant change in his or her cognitive state. Therefore, it would be advantageous to produce an interactive cognitive testing system capable of testing and assessing a user's cognitive state and taking appropriate action. Furthermore, the advantageousness of such a system would be enhanced by incorporating wireless communication capability to maintain the desired interactivity as a user moves and travels.

The present invention is directed to overcoming one or more of the problems set forth above.

### SUMMARY OF THE INVENTION

One aspect of the invention generally pertains to an interactive system and method that tests and assesses a user's cognitive state and/or a change thereto and is capable of taking appropriate action in response to that assessment.

Another aspect of the invention is to provide an interactive cognitive efficiency testing and assessment system and method incorporating the ability to communicate wirelessly.

Yet another aspect of the invention is to provide an interactive cognitive efficiency testing and assessment system and method that is capable of utilizing a variety of testing batteries and/or biometric sensors.

Another aspect of the invention is to provide a cognitive testing system with the ability to communicate with external devices or networks, such as the Internet, local area networks, wide area networks, satellites, or cellular networks, without a wired connection. An assessment and response center is either integrated into the system or is located in a remote centralized or regional facility that the system accesses via the wireless communication module.

In one embodiment of the invention, there is provided a wireless, interactive cognitive testing system that includes a testing module having an input device, a memory, and at least one cognitive test battery stored in the memory; a wireless communication module; and an assessment and response module.

In another embodiment of the invention, there is provided a wireless, interactive cognitive testing system that includes a testing module having an input device, a memory, and at least one cognitive test battery stored in the memory; a biometric testing module; a wireless communication module; and an assessment and response module.

An associated method of wireless, interactive cognitive testing in a human patient includes the steps of providing a testing module having an input device, a memory, and at least one cognitive test battery stored in the memory, and conducting at least one cognitive test with the cognitive test battery to produce at least one test result; and transmitting the test result via a wireless communication module to an assessment and response module.

These aspects are merely illustrative of the innumerable aspects associated with the present invention and should not be deemed as limiting in any manner. These and other aspects, features and advantages of the present invention will become apparent from the following detailed description when taken in conjunction with the referenced drawings.

### BRIEF DESCRIPTION OF THE DRAWINGS

Reference is now made more particularly to the drawings, which illustrate the best presently known mode of carrying out the invention and wherein similar reference characters indicate the same parts throughout the views.

FIG. 1 is a schematic diagram of an interactive cognitive testing and assessment system according to one embodiment of the present invention.

FIG. 2 is a schematic diagram of an interactive cognitive testing and assessment system according to another embodiment.

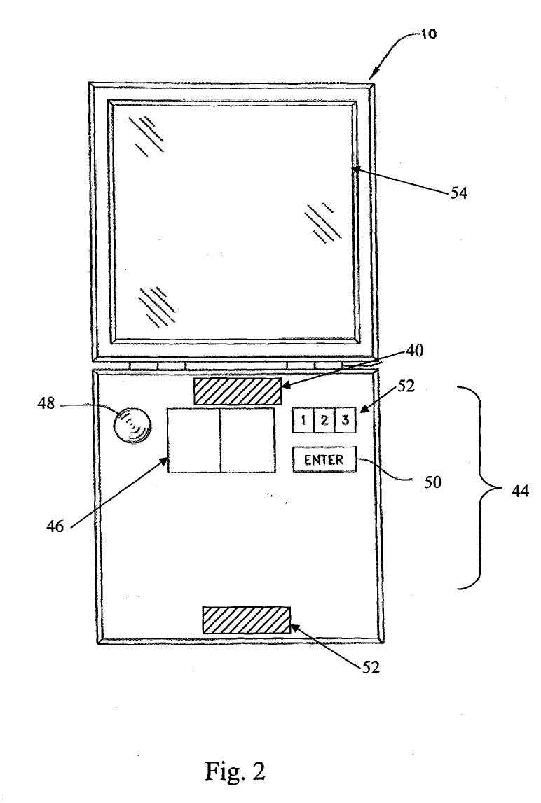

FIG. 3 is a schematic diagram of a system incorporating an interactive cognitive testing and assessment system according to another embodiment.

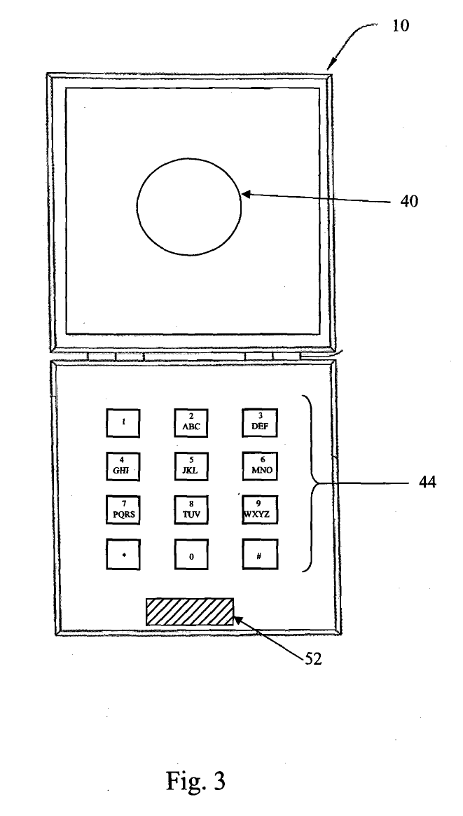

FIG. 4 is a schematic diagram of a biosensor for incorporation into an interactive cognitive testing and assessment system.

FIG. 5 is a flow chart illustrating the steps and sequence for a means of measuring cognitive efficiency and change in cognitive efficiency.

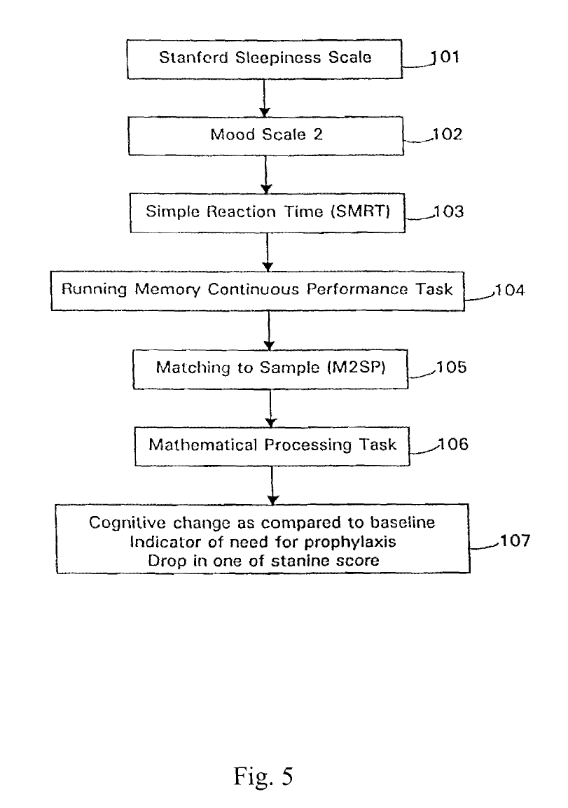

### DESCRIPTION OF THE PREFERRED EMBODIMENTS

In the following detailed description numerous specific details are set forth in order to provide a thorough understanding of the invention. However, it will be understood by those skilled in the art that the present invention may be practiced without these specific details. For example, the invention is not limited in scope to the particular type of industry application depicted in the figures. In other instances, well-known methods, procedures, and components have not been described in detail so as not to obscure the present invention.

The present system includes a system 10 having a testing module 12 and a wireless communication module 14. The testing module 12 provides a cognitive assessment test delivery device 16 and an input device 18. The wireless communication module 14 allows the system 10 to communicate with external devices or networks 20, such as the Internet, wireless local or wide area networks, or cellular networks, without a wired connection. In a preferred embodiment, the system also includes at least one assessment and response center 22 with which the system 10 connects via the wireless communication module 14.

In general terms, at least one cognitive test battery 100 is made available to a user via the assessment test delivery device 16. The user responds to those tests using the input device 18 as described in more detail below. The wireless communication module 14 transmits the user's responses and/or resulting cognitive test scores to the assessment and response center 22, where those responses/scores are evaluated and further action taken as needed. However, it is contemplated within the scope of the present invention that the assessment of scores and commencement of corresponding action may be accomplished within the system 10 itself rather than in communion with an external device or server.

In alternate embodiments, the wireless communication module 14 includes a cellular transmitter/receiver 24, a wi-fi transmitter/receiver 26, a wireless broadband modem 28, a Bluetooth transmitter/receiver 30, or an infrared (“IR”) transmitter/receiver 32. In preferred embodiments, the module 14 will include a combination of these communication means to provide a user with alternate forms of connecting to a device or network 20 in order to access the assessment and response center 22 or to seek assistance. For example, in a system 10 having both a cellular transmitter/receiver and wi-fi, a user would be able to call the assessment and response center 22 via an accessible cellular network or access the center via the internet through an available wireless network. The addition of a Bluetooth or IR transmitter/receiver would enable the user to further communicate with a local assessment and response device, or other compatible equipment (as discussed below) directly.

In another embodiment, the wireless communication module 14 may include a satellite telephone transmitter/receiver 34 for use when in locations having neither cellular nor wide/local area wireless networks.

The assessment and response center 22 may similarly take various forms in alternate embodiments. In a preferred embodiment the center 22 comprises a single centralized 36, or multiple regional 38, facilities that posses at least one computer server and/or human operator accessible by either or both the Internet or telephone. In another embodiment, the center 22 is entirely automated. As referenced above, the center 22 may also be directly incorporated within the system 10 itself.

Multiple options also exist for the test delivery device 16 and input device 18. In one embodiment, the test delivery device 16 comprises a speaker 40 through which test voice cues are audiblized to a user. The user responds to those test cues by speaking into a microphone 42, which forms the input device 18.

In a variation of this embodiment, the user responds to the test cues by pressing buttons on a keypad 44, which serves as the input device 18. The key pad has a built-in set of two mouse buttons 46, a start/stop or on/off button 48, an enter key 50, and Mood Scale 2 keys 1, 2 and 3(52). As used herein the terms “buttons” and “keys” are intended to mean the same thing. The keypad 44 may take the form of a standard numeric telephone keypad, with the above-described functions being incorporated therein as alternate key operations. In a basic and advantageously economical version of this embodiment, the system 10 is a standard cellular telephone. The user places a call to the assessment and response center 22 to communicate with either a human or automated attendant and initiate testing. The attendant delivers the verbal test cues, to which the user responds. The attendant evaluates the user's responses and determines whether further action is appropriate.

In another variation, the test delivery device 16 is formed by a screen 54 for viewing visual cognitive test stimuli.

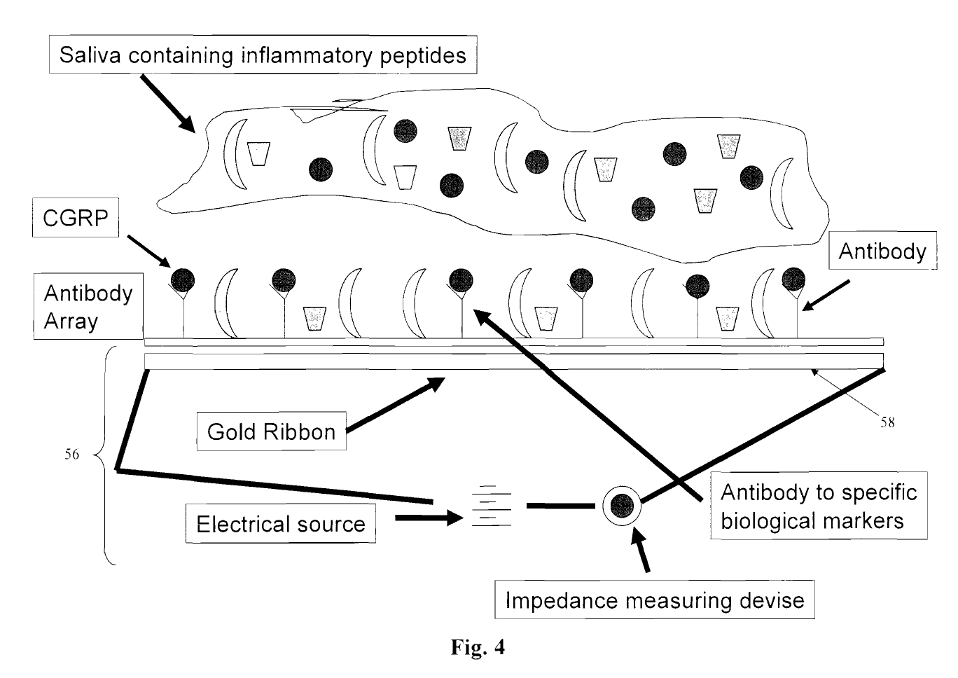

In yet another variation a biometric sensor 56 is incorporated into the system 10. The biosensor 56, as schematically illustrated in FIG. 4, consists of a substrate 58 that is coated with D-poly lysine on which antibodies to various biological markers are randomly arranged. In a preferred embodiment, the substrate 58 comprises a gold ribbon. When saliva is applied to the biosensor 56, a biological marker present in the saliva will bind to its correlating antibody. The interaction of biological marker and antibody alters the electrical impedance of the substrate 58. This change in impedance can be readily measured and converted into a transmittable electrical signal. It is possible to use various salivatory proteins with this methodology to provide measurement and identification of biological markers critical in diagnosis, staging of disease progression, and treatment of many head and facial pain disorders. In particular, the biosensor can accept a sample of saliva from which important biological markers such as c-reactive protein, CGRP, endorphins, enkephalins, human growth factors, electrolytes, prostagladins, and gluten proteins are measured and correlated to serum levels and disease risk and progression. The system can be used to accurately measures changes in these biological markers.

The biosensor 56 relies on a method consisting of providing a series of antibodies to several biological markers on a test strip in a known manner, including inflammatory peptides, proteins, human growth factors, endorphins, enkephalines, prostaglandins, and a variety of cytokines. A sample of saliva is applied to the series of antibodies by a user. If the anticipated biological markers are present in the sample of saliva, those markers bind to their correlating antibodies thereby providing an indicator of the presence and amount of a particular biological marker in the patient's saliva upon evaluation of the test strip. A baseline sample may be taken and compared to subsequent samples in order to determine changes in the levels of specific biological markers in the user over time.

The biosensor 56 is sensitive to biological markers such as calcitonin-gene-related peptide, vasoactive intestinal peptide, neurokinin A and B, substance P, c-reactive protein, amylase, IgG, IgA, nitric oxide, prostaglandins, and histamine.

A preferred embodiment of the system incorporates an improvement to the Automated Neuropsychological Assessment Metrics (ANAM). ANAM is a set of standardized batteries of cognitive tests, modified by neuropsychologists in the U.S. Armed Forces for precise measurement of cognitive processing efficiency of military personnel. The tests assess sustained concentration and attention, mental flexibility, spatial processing, cognitive processing efficiency, mood, arousal/fatigue level, and short-term, long-term and working memory. ANAM is now in the public domain. The most recent version is ANAM V3.11a/96 which includes the following battery of tests:
1. Subject Demographics Form
2. Stanford Sleepiness or Sleep/Fatigue Scale
3. Mood Scale 2
4. Simple and Two-Choice Reaction Time
5. Sternberg Memory Search Tasks
6. Running Memory Continuous Performance Task
7. Mathematical Processing Task
8. Digit Set Comparison Task
9. Logical Reasoning-Symbolic
10. Tower of Hanoi (Tower Puzzle)
11. Stroop Color/Word Interference
12. Code Substitution (Letter/Symbol Comparison)
13. Code Substitution (Immediate and Delayed Recall)
14. Spatial Processing Task (Simultaneous)
15. Matching to Sample
16. Tapping (Left and Right Index Finger)
17. Modified Orientation and Amnesia Test

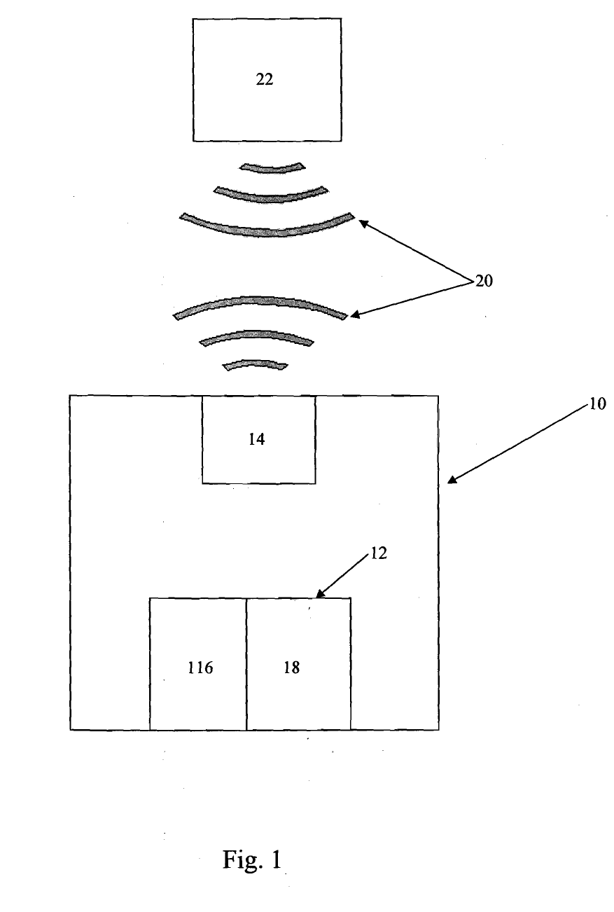

The improvement to ANAM used in one embodiment of the present system is disclosed in U.S. Pat. Nos. 6,066,092 and 6,416,472, the disclosures of which are expressly incorporated herein by reference. FIG. 1 shows the sequence of a cognitive efficiency measurement method used in the system, as follows:
1. Simple Reaction Time (SMRT), 103
2. Running Memory Continuous Performance Task (CPT), 104
3. Matching to Sample (M2SP), 105
4. Mathematical Processing Task (MATH), 106

In an alternate embodiment only two of the ANAM tests are used in the system:
1. Simple Reaction Time (SMRT), 103
2. Running Memory Continuous Performance Task (CPT), 104

Also included are two preliminary measures of alertness and mood that are also part of the ANAM:
1. Stanford Sleepiness Scale, 101
2. Mood Scale 2, 102.

The first step 101 is Stanford Sleepiness Scale which consists of seven statements that describe the present state of alertness or sleepiness and are numbered from one to seven, with one being highly alert and seven being close to sleep. Individuals rate their level of alertness prior to taking the first subtest of the battery. It provides a way to monitor fatigue over the course of repeated measures. Subjective ratings may be correlated with measured performance.

The second step 102 is Mood Scale 2 which consists of a list of thirty-six adjectives that are rated on a three-point scale. Using mouse button 16 participants respond to each adjective by indicating “yes,” “moderately,” or “no,” based on how they feel at the present time. The Mood Scale 2 categories include anger, happiness, fear (anxiety), depression, activity, and fatigue.

The third step 103 is Simple Reaction Time (SMRT) which presents a simple stimulus on the screen (*). In response, the individual presses the mouse button 16 each time the stimulus appears. The Reaction Time measures the speed of the motor response, the peripheral nerve conduction velocity. This represents the “hardware” of the nervous system in terms of input, followed by motor response. Actual cognitive processing time is not involved in this test.

The fourth step 104 is Running Memory Continuous Performance Test (CPT) which is a continuous letter comparison task. A randomized sequence of upper-case letters, A through Z, is presented one at a time in the center of the computer screen 14. The person presses button 16 if the letter on the screen matches the letter that immediately preceded it; and different button 18 if the letter on the screen is different than the immediately preceding letter. The task lasts approximately five minutes. The CPT was specifically designed to assess components of memory, attention, efficiency and consistency. This task is forced paced, with individuals having only a brief time in which to respond.

The fifth step 105 is Matching to Sample (M2SP) and consists of a number of trials that begins with a first design being presented in the center of the screen 14 for three seconds, followed by a showing that contains two designs. The person matches one of the two designs with the first design or sample by pressing the appropriate button 16 or 18. The design is a 4.times.4 checkerboard and varies by the number of cells that are shaded from one cell through twelve cells.

The sixth step 106 is Mathematical Processing (MATH) and involves arithmetic problems presented in the middle of the screen 14. Working from left to right, the person solves the addition and subtraction and decides if the answer is greater or less than the number 5.

As discussed above, these tests may be programmed directly into the memory of the system 10 or delivered when the user connects to the assessment and response center 22. As a result of the system's wireless communication module 14, the test battery(ies) programmed into the system 10 may be updated or even replaced automatically when the system 10 communicates with the assessment and response center 22.

As previously referenced, the assessment and response center 22 is capable of taking appropriate action depending on the user's responses/test scores. In particular, if the user's responses indicate a dramatic decline in cognitive function, emergency medical assistance can be automatically summoned to the user's location. This function may be aided by the incorporation of a global positioning satellite (GPS) receiver 60 within the system. The GPS receiver 60 provides a means of identifying the device's (and hence the user's) current coordinates to direct the required assistance promptly to the user with reduced delay.

Alternatively, the assessment and response center 22 can transmit a signal via the wireless communication module 14 to any compatible motorized vehicles or machines in the immediate vicinity of the user directing those machines to stop operation in order to reduce the likelihood of injury to the user or other persons. The incorporation of a Bluetooth transmitter/receiver 30 is particularly useful for this purpose.

It is contemplated that a system user may at times be in a remote location that does not have a consistently available cellular or wireless network. In the embodiments in which the assessment and response center 22 is located in a centralized or remote facility, the system 10 advantageously stores recently entered responses and calculated test scores in the event a connection with the assessment and response center 22 can not be made. In these cases, the central processor of the system 10 is programmed to automatically attempt reconnection with the assessment and response center 22 at regular timed intervals via all available networks until a connection can be made, at which time all recent responses and test scores are then communicated to the assessment and response center 22 for evaluation and appropriate action. In this particularly advantageous embodiment, which may also include the GPS receiver 60 described above, the system is capable of summoning help directly to a user who has suffered dangerously reduced cognitive functioning.

The preferred embodiments of the invention have been described above to explain the principles of the invention and its practical application to thereby enable others skilled in the art to utilize the invention in the best mode known to the inventors. However, as various modifications could be made in the constructions and methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by the above-described exemplary embodiment, but should be defined only in accordance with the following claims appended hereto and their equivalents.

I claim:

1. A wireless, interactive cognitive testing system, comprising:
a testing module, said testing module having an input device, a memory, and at least one cognitive test battery stored in said memory,
a wireless communication module, and
an assessment and response module.

2. The wireless, interactive cognitive testing system of claim 1, further comprising a biometric testing module.

3. The wireless, interactive cognitive testing system of claim 2, wherein said biometric testing module comprises:
a biosensor comprising a substrate having an electrical impedance and a coating;
at least a first antibody corresponding to at least a first biological marker on said coating, said antibody operative for binding with said biological marker present in a sample of saliva from a human patient; and
a microprocessor in communication with said biosensor, said microprocessor operable for accepting an electrical signal from said biosensor and producing test data corresponding to said electrical signal.

4. The wireless, interactive cognitive testing system of claim 3, wherein said biological marker is selected from the group consisting of inflammatory peptides, proteins, human growth factors, endorphins, enkephalins, prostaglandins, and cytokines.

5. The wireless, interactive cognitive testing system of claim 3, wherein said biological marker is selected from the group consisting of calcitonin-gene-related peptide, vasoactive intestinal peptide, neurokinin A and B, substance P, c-reactive protein, amylase, IgG, IgA, nitric oxide, prostaglandins, and histamine.

6. The wireless, interactive cognitive testing system of claim 3, wherein said coating comprises D-poly lysine.

7. The wireless, interactive cognitive testing system of claim 3, wherein said substrate comprises a gold ribbon.

8. The wireless, interactive cognitive testing system of claim 1, wherein said cognitive test battery comprises at least one test from the Automated Neuropsychological Assessment Metrics (ANAM).

9. The wireless, interactive cognitive testing system of claim 8, wherein said cognitive test battery consists of a Simple Reaction Time test, a Running Memory Continuous Performance test, a Matching to Sample test, and a Mathematical Processing test.

10. The wireless, interactive cognitive testing system of claim 8, wherein said cognitive test battery consists of a Stanford Sleepiness Scale test and a Mood Scale 2 test, a Simple Reaction Time test, a Running Memory Continuous Performance test, a Matching to Sample test, and a Mathematical Processing test.

11. The wireless, interactive cognitive testing system of claim 1, wherein said assessment and response module is housed with said testing module and said wireless communication module.

12. The wireless, interactive cognitive testing system of claim 1, wherein said assessment and response module is located remotely from said testing module and said wireless communication module.

13. The wireless interactive cognitive testing system of claim 12, wherein said assessment and response module comprises a centralized facility.

14. The wireless, interactive cognitive testing system of claim 12, wherein said assessment and response module comprises multiple regional facilities.

15. The wireless, interactive cognitive testing system of claim 12, wherein said assessment and response module comprises at least one computer server.

16. The wireless, interactive cognitive testing system of claim 12, wherein said assessment and response module comprise at least one human operator.

17. The wireless, interactive cognitive testing system of claim 1, wherein said wireless communication module comprises at least one of the group consisting of a cellular transmitter/receiver, a wi-fi transmitter/receiver, a wireless broadband modem, a Bluetooth®, transmitter/receiver, and an infrared transmitter/receiver.

18. The wireless, interactive cognitive testing system of claim 1, wherein said wireless communication module comprises a satellite telephone.

19. The wireless, interactive cognitive testing system of claim 1, further comprising a GPS module.

20. A wireless, interactive cognitive testing system, comprising:
a testing module, said testing module having an input device, a memory, and at least one cognitive test battery stored in said memory;
a biometric testing module;
a wireless communication module; and
an assessment and response module.

21. A wireless, interactive cognitive testing system, comprising:
a testing module, said testing module having an input device, a memory, and at least one cognitive test battery comprising at least one test from the Automated Neuropsychological Assessment Metrics (ANAM) stored in said memory;
a wireless communication module; and
an assessment and response module.

22. A method of wireless, interactive cognitive testing in a human patient, comprising the steps of:
providing a testing module having an input device, a memory, and at least one cognitive test battery stored in said memory, and
conducting at least one cognitive test with said cognitive test battery to produce at least one test result; and
transmitting said test result via a wireless communication module to an assessment and response module.

23. The method of wireless, interactive cognitive testing of claim 22, further comprising the steps of:
providing a biosensor comprising a substrate having an electrical impedance and a coating, at least a first antibody corresponding to a first biological marker being arranged on said coating;
applying a sample of saliva from said human patient to said biosensor;
measuring a change in said electrical impedance of said biosensor substrate resulting from binding of said antibody with said biological marker present in said saliva sample:
converting said change in said electrical impedance of said biosensor substrate into a transmittable electrical signal;
transmitting said electrical signal to a microprocessor in communication with said biosensor;
producing test data in said microprocessor corresponding to said electrical signal; and
transmitting said test data via said wireless communication module to said assessment and response module.
